(19) United States
(12) Patent Application Publication
(10) Pub. No.: US 2003/0044879 A1
(43) Pub. Date: Mar. 6, 2003

(54) Method and device for forming a concentration gradient for chemotactic evaluation through use of laminar flow

(76) Inventors: Michael Beyer, San Jose, CA (US); Ulrich Kruhne, Kobenhavn (DK); Thomas Ahl, Cupertino, CA (US)

Correspondence Address: REED & ASSOCIATES, 800 MENLO AVENUE, SUITE 210, MENLO PARK, CA, 94025, US

(21) Appl. No.: 09/941,950

(22) Filed: Aug. 28, 2001

(51) Int. Cl. C12Q 1/02; C12M 1/34

(52) U.S. Cl. 435/29; 435/287.9

(57) ABSTRACT

The invention relates to methods and devices for forming a concentration gradient of a reagent for use in chemotactic evaluation. A flow passage is provided and defined at least in part by a substrate having a target region on a surface thereof. A concentration gradient of a reagent is formed over the target region by controlled delivery of a fluid containing the reagent in laminar flow through the flow passage and over the target region. The concentration gradient is suitable for chemotactic evaluation. Typically, the inventive methods and devices are employed to evaluate the chemotactic interaction between a candidate compound and a monolayer of immobilized cells.

### TECHNICAL FIELD

The present invention relates to devices and methods for forming a reagent concentration gradient over a substrate surface through use of laminar flow. More specifically, the invention relates to devices and methods that provide for such gradient formation to carry out cell-based chemotactic evaluation.

### BACKGROUND

Chemotaxis is broadly defined as the orientation or movement of an organism or cell in response to a chemical concentration gradient. Chemotactic assays are widely used procedures in medical, biological, pharmaceutical and toxicological research. Such assays may be employed, for example, to research chemoattractants, which are mediators that activate cell adhesion and motility and direct cell migration through formation of a concentration gradient. In addition, chemotactic assays may be employed to study substances that inhibit chemoattractive activity. Often, chemotactic assays are employed to determine the effect of a chemical agent on the inflammatory process.

Currently used chemotactic assay procedures derive primarily from that originally developed by S. Boyden in 1962. See Boyden, (1962) “The Chemotactic Effect of Mixtures of Antibody and Antigen on Polymorphonuclear Leucocytes,” J. Exp. Med. 115:453-466. Generally, the procedure involves placing a suspension of cells in an upper chamber and a candidate chemoattractant in a lower chamber, wherein the chambers are separated by a porous membrane, typically of nitrocellulose or polycarbonate, that serves as a filter. The membrane is selected to confine the cells in the upper chamber in the absence of a force inducing the cells to migrate through the membrane. In some instances, the porous membrane may be coated with a protein such as collagen. The candidate chemoattractant diffuses into the upper chamber, thereby forming a concentration gradient across the membrane. After a period of time, cells on the upper membrane surface are carefully removed. The remaining cells within the porous membrane are then fixed and stained. Through microscopy, the porous membrane is examined to manually count the number of cells appearing on the underside of the membrane. The presence of cells having traveled through the membrane to the underside indicates a positive chemotactic response. In addition or in the alternative, the number of cells that accumulate in the lower chamber may be counted.

Variations of this technique have been described in a number of patents. For example, U.S. Pat. No. 4,912,057 to Guirguis et al. describes a chemotactic assay instrument that includes a bottom plate, a seal means mounted to the bottom plate, and an intermediate plate mounted on the other side of the seal means and spaced by the seal means from the bottom plate. The intermediate plate defines a plurality of wells to hold a sample. A porous membrane is mounted to the intermediate plate and serves to space a second intermediate plate from the first intermediate plate. A second seal means is mounted to the second intermediate plate, and a top plate is mounted to the second seal means. The top plate is spaced by the second seal means from the second intermediate plate. Also provided is a means to hold the plates together in a sealed relationship.

Similarly, U.S. Pat. No. 5,514,555 to Springer et al. describes a method for detecting or measuring lymphocyte chemotaxis. The method involves detecting or measuring the transmigration of lymphocytes completely through a porous membrane in a direction (a) toward increased levels of a known or suspected lymphocyte chemoattractant, and (b) from a first surface of the porous membrane toward an opposite, second surface of the porous membrane. The membrane is described as a microporous membrane having an endothelial cell monolayer on the first surface. The presence and extent of said transmigration of lymphocytes completely through the porous membrane indicates the presence and extent, respectively, of lymphocyte chemotaxis.

There are several disadvantages to these procedures. While they involve the formation of a gradient, control of gradient formation and the gradient formed thereby is not easily achieved. Thus, assays employing such procedures are only able to provide information relating to cellular response to the presence of a gradient, not cellular response as a result of any specific aspect of the gradient. In addition, these procedures typically require a relatively large quantity of cells and reagent; such a requirement are a drawback in instances where cells and reagents are rare or expensive.

Furthermore, these procedures involve a number of time-consuming and tedious steps. As discussed above, such methods usually require a dislodging process to remove the non-migrated cells from the porous membrane before the migrated cells can be fixed and stained for counting. Such a dislodging process involves a high level of expertise and care in handling since it is important to avoid disturbing cells located on the underside of the membrane. Cell counting is another lengthy and cumbersome task. If all fixed cells were counted, the procedure would be prohibitively time-consuming. Thus, only cells selected at random from representative areas are ordinarily examined and counted. This, of course, involves a possible compromise to accuracy and reproducibility, as such a protocol tends to exhibit significant variability. The count itself is also highly subjective because it requires the exercise of judgment in determining whether to count cells that have only partially migrated across the porous membrane. Notably, the fixation and staining steps kill the cells. Also important to note is that the processes associated with fixation, which employ porous membranes and/or suspension cultures, could limit the types of cells that are available for evaluation of chemotactic properties.

Since the fixation and staining processes kill the cells, these procedures are not, as a rule, easily adapted to kinetic or time-dependent studies of chemotactic response within the same cell sample. In order to determine a time-dependent chemotactic response of a cell to a candidate chemoattractant, it is typically necessary to run multiple cell samples under varying time constraints. The number of required samples and experiments are greater due to low protocol accuracy. Since multiple samples as well as positive and negative controls are required to obtain reliable data, a single kinetic study may result in use of a large number of porous membranes, each requiring labor-intensive and time-consuming examination. To overcome this drawback, U.S. Pat. No. 5,601,997 to Tchao describes a non-destructive chemotaxis assay procedure involving a technique similar to that described above, except that the cells are labeled with a fluorescent dye and a radiation opaque membrane is employed. By monitoring the fluorescence in the chamber containing the candidate chemoattractant, kinetic studies may be executed. It should then be evident that while this procedure provides a non-destructive chemotactic assay that allows for kinetic evaluation, it is still subject to the other disadvantages associated with ordinary chemotaxis techniques that employ a porous membrane.

In addition, a number of patents describe devices that contain a fluid in which a gradient is formed. For example, U.S. Pat. No. 3,449,938 to Gidding describes using a gradient to separate fluid materials. As another example, U.S. Pat. No. 5,716,852 to Yager et al. describes a T-sensor channel fluidic device that allows for the detection of analyte particles in a sample stream when such particles diffuse into an indicator stream. Similarly, U.S. Pat. No. 6,091,502 to Weigl et al. describes an optical detection device for performing spectral measurements in a flow cell in which a gradient may be formed. However, none of these patents describe the formation of a gradient over a target region of a substrate surface in order to carry out chemotactic evaluation.

Thus, there is a need for alternative devices and methods that perform chemotactic evaluation involving the formation of a chemical gradient over a target region of a substrate surface, preferably employing small quantities of reagents and/or cells. Such devices and methods should be capable of controlling the production of these concentration gradients, which are required for chemotactic evaluation.

### SUMMARY OF THE INVENTION

Accordingly, it is an object of the present invention to overcome the above-mentioned disadvantages of the prior art by providing methods and devices that form a concentration gradient of a reagent over a target region of a substrate surface by controlled delivery of a fluid containing the reagent in laminar flow for use in a chemotactic evaluation.

Additional objects, advantages, and novel features of the invention will be set forth in part in the description that follows, and in part will become apparent to those skilled in the art upon examination of the following, or may be learned through routine experimentation upon practice of the invention.

In one embodiment, the invention relates to a method for forming a concentration gradient of a reagent for use in a chemotactic evaluation. This method provides for a flow passage defined at least in part by a substrate having a target region on a surface thereof. A concentration gradient of a reagent is formed over the target region by controlled delivery of a fluid containing the reagent in laminar flow through the flow passage and over the target region. By immobilizing a cell (or a plurality of cells, preferably as a monolayer) on the target region and employing a candidate compound as the reagent, the method may be used to carry out chemotactic evaluation.

There are a number of techniques that can be employed to form a reagent concentration gradient over the target region through controlled delivery of a reagent-containing fluid in laminar flow. Generally these techniques may be either diffusion or non-diffusion based. For diffusion-based processes, diffusion may occur from a fluidic lane/layer into an adjacent lane/layer, from a fluidic lane to an adjacent carrier sheath, or from a reagent source upstream of a target region.

Specifically, one diffusion-based technique involves introducing a plurality of fluids, each fluid introduced through an inlet to form a lane downstream from each inlet in contiguous laminar flow through the flow passage, wherein at least one fluid contains the reagent. The reagent is allowed to diffuse across one or more lanes to form a concentration gradient over the target region. This technique allows the formation of static or dynamic concentration gradients.

Another technique involves maintaining a carrier fluid in contiguous laminar flow at a selected flow rate through the flow passage and over the target region. A stream containing a reagent is introduced through an inlet into the carrier fluid upstream from the target region at a flow rate appropriate to the selected flow rate of the carrier fluid, which allows the reagent to diffuse downstream in the carrier fluid to form a concentration gradient over the target region. This technique also allows the formation of static or dynamic concentration gradients.

A further technique involves providing a source of reagent located upstream from the target region, which is adapted to release reagent into the carrier fluid as the carrier fluid flows over the reagent source. A carrier fluid is maintained in contiguous laminar flow at a selected flow rate through the flow passage to contact the reagent source such that reagent is released into the carrier fluid to form a concentration gradient over the target region.

Yet another technique involves sweeping a hydrodynamically focused stream of fluid over the target region while simultaneously ensuring correspondence of the concentration of the reagent in the stream to a predetermined concentration profile. Depending on how the stream is swept and the desired gradient, a constant or varying reagent concentration may be used in the stream.

In some instances, the surface reactive sites on the target region may react with the reagent in the fluid flowing over the target region. As a result, the reaction may result in the formation of an immobilized reagent gradient on the target region.

In another embodiment, the invention relates to a device for forming a concentration gradient of a reagent for use in a chemotactic evaluation. The device comprises a flow passage defined at least in part by a substrate having a surface and a target region thereon, as well as a means for forming a concentration gradient of a reagent over the target region by controlled delivery of a fluid containing the reagent in laminar flow through the flow passage. The concentration gradient is suitable for chemotactic evaluation.

One such means for forming a concentration gradient over a target region comprises maintaining a carrier fluid in contiguous laminar flow at a carrier flow rate through the flow passage and over the target region. An inlet in fluid communication with a reagent source and upstream from the target region is provided, such that a stream of reagent may be introduced through the inlet at a flow rate appropriate for diffusion in the carrier, thus forming a concentration gradient suitable for chemotactic evaluation over the target region.

Another means for forming a concentration gradient over a target region comprises a plurality of inlets, each inlet located upstream from the target region. An introduction means for a plurality of fluids is provided such that each fluid flows from a fluid source through an inlet to form a lane downstream therefrom, each fluid also exhibiting contiguous laminar flow through the flow passage. Each fluid contains a concentration of the reagent, and the flow rates of the fluids are selected to allow the reagent to diffuse across one or more lanes to form a concentration gradient suitable for chemotactic evaluation over the target region.

Still another means for forming a concentration gradient over a target region comprises a means for maintaining a carrier fluid in contiguous laminar flow at a carrier flow rate through the flow passage and over the target region. A source of reagent located upstream from the target region is also provided to release reagent into the carrier fluid as the carrier fluid contacts the reagent source, creating a concentration gradient suitable for chemotactic evaluation over the target region.

A further means by which to form a concentration gradient over a target region comprises sweeping a hydrodynamically focused stream of fluid over the target region while simultaneously ensuring that the reagent concentration in the stream fits a predetermined profile.

In yet another embodiment, the invention relates to a method for producing a stream of fluid having a predetermined reagent concentration profile. The method provides for a fluid vessel having a cavity extending from an inlet opening to an outlet opening. Fluids containing different concentrations of the reagent are loaded in sequence though the inlet opening into the cavity, wherein the sequence is selected to correspond to a predetermined reagent concentration profile. Then, the loaded fluid is expelled through the outlet of the vessel to produce a stream of fluid that exhibits the predetermined reagent concentration profile. When a gradual concentration profile is desired, the method calls for passage of sufficient time to allow diffusion of the reagent within the cavity to correspond to the predetermined reagent concentration profile.

In a further embodiment, the invention relates to a method for carrying out a cellular assay. The method provides for a flow passage defined at least in part by a substrate having a cell immobilized on a surface thereof. A concentration gradient of a reagent is formed over the cell by controlled delivery of a fluid containing the reagent in laminar flow through the flow passage and over the target region. The method further involves the detection of a cellular response to the concentration gradient, such as chemotaxis.

In a yet further embodiment, the invention relates to a method for carrying out a cellular assay. The device comprises a flow passage defined at least in part by a substrate having a cell immobilized on a surface thereof, a means for forming a concentration gradient of a reagent over the cell by controlled delivery of a fluid containing the reagent in laminar flow through the flow passage and over the target region, and a detector for detecting cellular response to the concentration gradient.

### BRIEF DESCRIPTION OF THE DRAWINGS

FIGS. 1A-1C, collectively referred to as FIG. 1, illustrate a method for forming a gradient over a surface that does not employ laminar flow. FIG. 1A illustrates in exploded view a slide and cover slip assembly for forming a gradient over the slide surface. FIG. 1B illustrates in cross-sectional view the formed assembly. FIG. 1C illustrates in top view the assembly having a reagent concentration gradient formed therein.

FIGS. 2A-2D, collectively referred to as FIG. 2, illustrate a device that may be employed to form a concentration gradient over a target region of a substrate surface. FIG. 2A illustrates the device in exploded view. FIG. 2B schematically illustrates the device in an assembled form. FIG. 2C schematically illustrates the formation of a gradient of over the target region using the device illustrated in FIG. 2C. FIG. 2D schematically illustrates the formation of a different gradient using a variation of the device illustrated in FIG. 2C.

FIGS. 3A and 3B, collectively referred to as FIG. 3, illustrate another device that may be employed to form a concentration gradient over a target region of a substrate surface. FIG. 3A illustrates the device in exploded view. FIG. 3B schematically illustrates the device in an assembled form and in operation.

FIGS. 4A and 4B, collectively referred to as FIG. 4, illustrate a device similar to that illustrated in FIG. 3 except that a reagent source is provided on a substrate source upstream from a target region. FIG. 4A illustrates the device in exploded view. FIG. 4B schematically illustrates the device having a gradient formed over a target region of a substrate surface.

FIGS. 5A-5D, collectively referred to as FIG. 5, illustrate a device that may be employed carry out the inventive method by sweeping a hydrodynamically focused stream over a target region of a substrate surface. FIG. 5A illustrates the device in exploded view. FIGS. 5B-5D schematically illustrate the device in assembled form, wherein a reagent lane formed from a hydrodynamically focused stream is swept over a target region of a substrate surface from one side wall to an opposing side wall.

### DETAILED DESCRIPTION OF THE INVENTION

Before the invention is described in detail, it is to be understood that, unless otherwise indicated, this invention is not limited to particular materials, components, or manufacturing processes, as such may vary. It is also to be understood that the terminology used herein is for purposes of describing particular embodiments only, and is not intended to be limiting.

It must be noted that, as used in the specification and the appended claims, the singular forms “a,” “an,” and “the” include plural referents unless the context clearly dictates otherwise. Thus, for example, reference to “a lane” includes a plurality of lanes, “a reagent” includes a mixture of reagents, “an inlet” includes a plurality of inlets, and the like.

In this specification and in the claims that follow, reference will be made to a number of terms that shall be defined to have the following meanings:

The term “array” used herein refers to a two-dimensional arrangement of features such as cells or molecular moieties on a substrate surface. Arrays are generally comprised of regular, ordered features, as in, for example, a rectilinear grid, parallel stripes, spirals, lanes, and the like, but non-ordered arrays may be advantageously used as well. An array differs from a pattern in that patterns do not necessarily contain regular and ordered features.

The term “cell line” as used herein refers to a permanently established cell culture that will proliferate indefinitely given appropriate fresh medium and space. While cell lines are readily available for some species such as those in the rodent family, and difficult to establish for other species such as humans, the term “cell line” as used herein is not limited to any particular species or cell type.

The term “chemotaxis” as used herein refers the movement of a cell in response to a chemical gradient. As used herein, the term may refer to the movement of an entire cell or a portion thereof, e.g., nucleus, cytoplasm, mitochondria, in relation to remaining portions of the cell.

The term “concentration” as used herein refers to the molar ratio of a substance to fluid volume in a stream. The substance may be entirely soluble, partially soluble, or insoluble in the fluid of the stream. Thus, the term “concentration profile” is used herein to refer to variations in the molar ratio of a substance to fluid volume in a stream, regardless of whether the substance is soluble in the fluid. For example, a stream of fluid that exhibits an increasing ratio of reagent to fluid volume is said to have a concentration profile that exhibits an increasing concentration. Similarly, a gradual concentration profile is one in which the concentration varies by progressive degrees rather than in a stepwise manner.

The term “fluid-tight” is used herein to describe the spatial relationship between two solid surfaces in physical contact such that fluid is prevented from flowing into the interface between the surfaces.

The term “gradient,” as in “concentration gradient” or “chemical gradient,” is used herein in its ordinary sense and refers to the variation of a parameter, e.g., concentration over a given distance. Gradients may be formed from simple or complex chemical structures. For example, chemicals that may form a gradient include, but are not limited to, biological molecules such as proteins, peptides, antibodies, cells, viral particles, sugars, proteoglycans, and lipids.

The terms “immobilize,” “immobilized,” and “immobilizing,” e.g., as in “immobilized cells,” are used herein to describe the fixation of a cell to a position on a substrate surface such that non-chemotactic movement of the cell does not occur. For example, an immobilized cell exposed to a laminar flow that exhibits a chemical gradient may not move in response to the fluid flow but may move in response to the chemical gradient.

The term “laminar flow” as used herein refers to fluid movement in the absence of turbulence, such that mixing of fluid components occurs solely or primarily as a result of diffusion. The Reynolds number associated with laminar flow described herein is typically about 0.1 to about 200, preferably about 1 to 20, and optimally about 2 to 10.

The term “lane” as used herein refers to one of a set of typical routes or courses along which a fluid travels or moves. While a lane may be bounded by one or more solid surfaces, a lane of fluid is bounded by at least another fluid, with which nondiffusional mixing does not occur. Thus, a reagent in one lane of fluid bounded by another lane may diffuse across the boundary between the lanes.

“Optional” or “optionally” as used herein means that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “primary cell” is used herein in its ordinary sense and refers to a cell taken directly from a living tissue that has not been immortalized. Primary cells may be derived from a number of sources such as from an in vivo or ex vivo organ culture. For example, primary cells may be taken from a liver biopsy, a fetus, or embryonic tissue.

The term “reagent” is used herein to refer to any substance that is used in a chemical or biochemical or biological reaction to detect, measure, examine, or produce other substances. Reagents may be contained in a fluid in solvated, partially solvated, or suspended form.

The term “substrate” as used herein refers to any material having a surface over which laminar fluid flow may occur. The substrate may be constructed in any of a number of forms such as wafers, slides and well plates, and membranes. Suitable substrate materials include, but are not limited to, supports that are typically used for cell handling, e.g.: polymeric materials (e.g., polystyrene, polyvinyl acetate, polyvinyl chloride, polyvinyl fluoride, polyacrylonitrile, polyacrylamide, polymethyl methacrylate, polytetrafluoroethylene, polyethylene, polypropylene, polybutylene, polyvinylidene fluoride, polycarbonate, polyimide and polyethylene teraphthalate); silica and silica-based materials; functionalized glasses; ceramics; and such substrates treated with surface coatings, polymeric and/or metallic compounds, or the like. While the foregoing support materials are representative of conventionally used substrates, it is to be understood that the substrate may in fact comprise any biological, nonbiological, organic and/or inorganic material, and may further have any desired shape, such as a disc, square, sphere, circle, etc. The substrate surface is typically but not necessarily flat, e.g., the surface may contain raised or depressed regions.

The term “surface modification” as used herein refers to the chemical, biological, and/or physical alteration of a surface by an additive or subtractive process to change one or more chemical and/or physical properties of a substrate surface or a selected location or region of a substrate surface. For example, surface modification may involve: (1) changing the wetting properties of a surface; (2) functionalizing a surface, i.e., providing, modifying, or substituting surface functional groups; (3) defunctionalizing a surface, i.e., removing surface functional groups; (4) otherwise altering the chemical composition of a surface, e.g., through etching; (5) increasing or decreasing surface roughness; (6) providing a coating on a surface, e.g., a coating that exhibits wetting properties that are different from the wetting properties of the surface; and/or (7) depositing particulates on a surface. Thus, for example, surface modification may involve providing a biologically derived coating on a surface, wherein the coating comprises a naturally occurring polymer such as a protein or peptide (e.g., collagen, fibronectin, albumin, fibrinogen, or thrombin), a saccharide (e.g., polymannuronic acid, polygalacturonic acid, dextran, or glycoaminoglycan), or a synthetic polymer (e.g., polyvinyl alcohol, acrylic acid polymers, and acrylic acid copolymers).

The term “target region” as used herein refers to a predefined two-dimensional area over which fluid is directed to flow. The target region is typically, but not necessarily, contiguous and may or may not have cells adhered thereto. The target region may exhibit any of a variety of surface properties as long as the surface properties are predetermined. In some instances, for example, the target region may be functionalized so as to have surface reaction sites that allow a reagent to be attached thereto. In other instances, the target region may be selected for its ability to repel certain reagents.

Thus, the invention relates generally to methods and devices that cause the formation of a reagent concentration gradient for use in chemotactic evaluation. These methods and devices involve providing a flow passage defined at least in part by a substrate having target region on a surface thereof and the formation of a reagent concentration gradient over the target region by controlled delivery of a fluid containing the reagent in laminar flow through the flow passage and over the target region. The invention also provides a number of means by which to form a reagent concentration gradient over a target region of a substrate surface by controlled delivery of a fluid in laminar flow through the flow passage. These inventive methods and devices are useful in carrying out chemotactic evaluation of cell movement in response to a candidate compound.

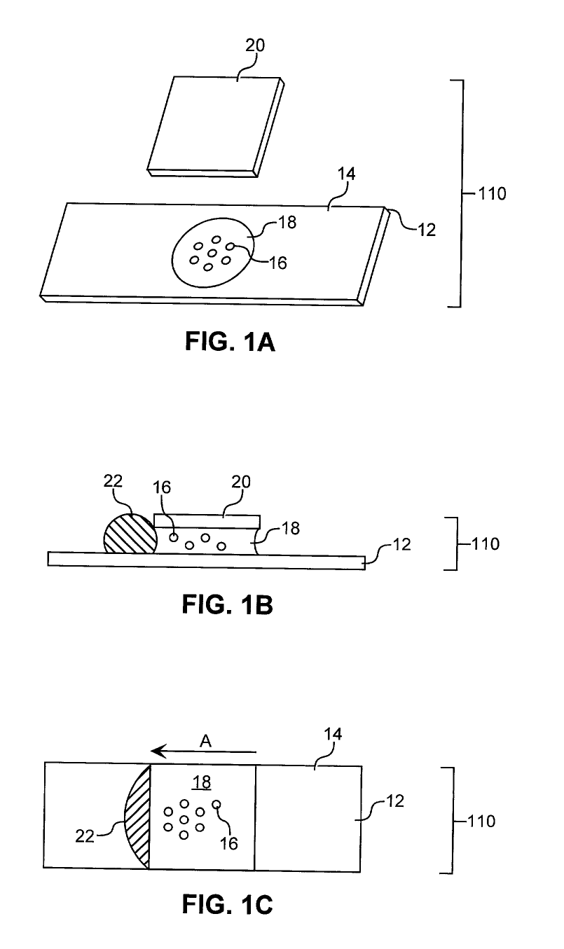

To illustrate the advantages of the invention, a method for forming a chemical gradient over a substrate surface without the use of laminar flow is provided to portray the disadvantages associated therewith. FIG. 1 schematically illustrates an assembly for forming a chemical gradient over a surface to carry out chemotactic evaluation of cells immobilized thereon. As with all figures referenced herein, in which like parts are referenced by like numerals, FIG. 1 is not necessarily to scale, and certain dimensions may be exaggerated for clarity of presentation. As shown in FIG. 1A, the assembly 10 includes a substrate 12, typically an ordinary glass slide having an upper surface 14. Live cells 16 and a carrier fluid 18 in sufficient quantity to submerge the cells are placed on the upper surface 14 of the substrate, and an ordinary cover slip 20 is placed over the cells and the carrier fluid. The carrier fluid is selected from a culturing medium to sustain the cells. As a result, the cover slip is free-floatingly located in opposing relation with the substrate, and the cells and the carrier fluid are interposed between the cover slip and the substrate.

In order to carry out a chemotactic assay, a chemical gradient is produced in the fluid in contact with cells by placing a source of reagent 22 in contact with the carrier fluid 18 at an edge of the cover slip, shown in FIG. 1B. As the reagent travels away from the source through diffusion, shown in FIG. 1C, a concentration gradient is formed in the carrier fluid. The reagent concentration is inversely proportional to the distance from the reagent source. Thus, reagent concentration increases in the carrier fluid direction indicated by arrow A. As a result, the cells are exposed to the concentration gradient. Subsequently, a chemotactic assay may be conducted by detecting whether the cells or portions thereof move in response to the gradient.

This assembly suffers from a number of disadvantages. For example, in order to survive, living cells, through ordinary biological processes, may extract nutrients and other essential chemicals from, and introduce waste products into, the surrounding medium. To sustain the living cells, then, the local medium in contact with the cells must provide a continuous supply of nutrients without accumulating a toxic concentration of waste products. This can be accomplished either by employing a large quantity of culturing medium, or by replenishing essential nutrient-containing fluid to the cells while simultaneously eliminating cell waste products. The above-described assembly, however, neither provides a large volume of culturing medium nor allows the carrier fluid to be easily replenished without removing the cover slip. Such removal would disturb any reagent gradient formed in the carrier fluid. In addition, once assembled, the user would effectively have no control over the formation of the gradient, since the formation of the gradient would be determined by diffusion of the reagent from the source. If the diffusion of the reagent occurred too slowly, the cells might die before the chemotactic assay could be completed. Furthermore, the gradient of reagent would vary over time and eventually cease to exist when the carrier fluid became either saturated with the reagent or uniform in reagent concentration. Thus, the aforementioned assembly would be incapable of maintaining a static concentration gradient.

The present invention, on the other hand, provides vastly improved control over gradient formation. As discussed above, the invention involves controlled delivery of a fluid containing the reagent in laminar flow through a flow passage to form a concentration gradient for use in a chemotactic evaluation. One technique for effecting controlled delivery of a fluid containing a reagent involves maintaining a carrier fluid in contiguous laminar flow at a selected flow rate through the flow passage and over the target region. A stream containing a reagent would be introduced through an inlet into a carrier fluid upstream from the target region at a flow rate appropriate for the reagent to diffuse downstream, such that a concentration gradient would form over the target region.

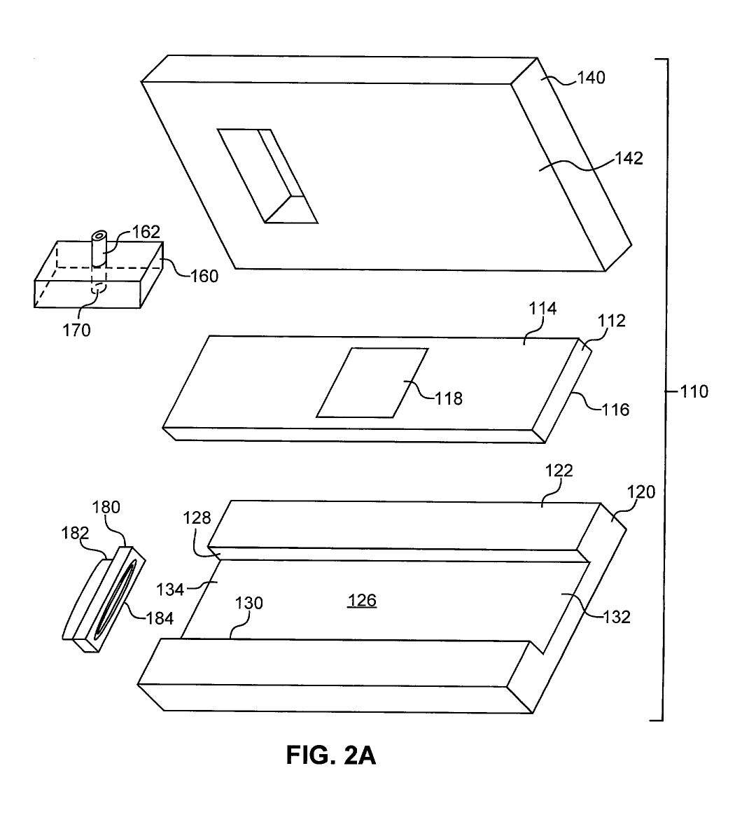

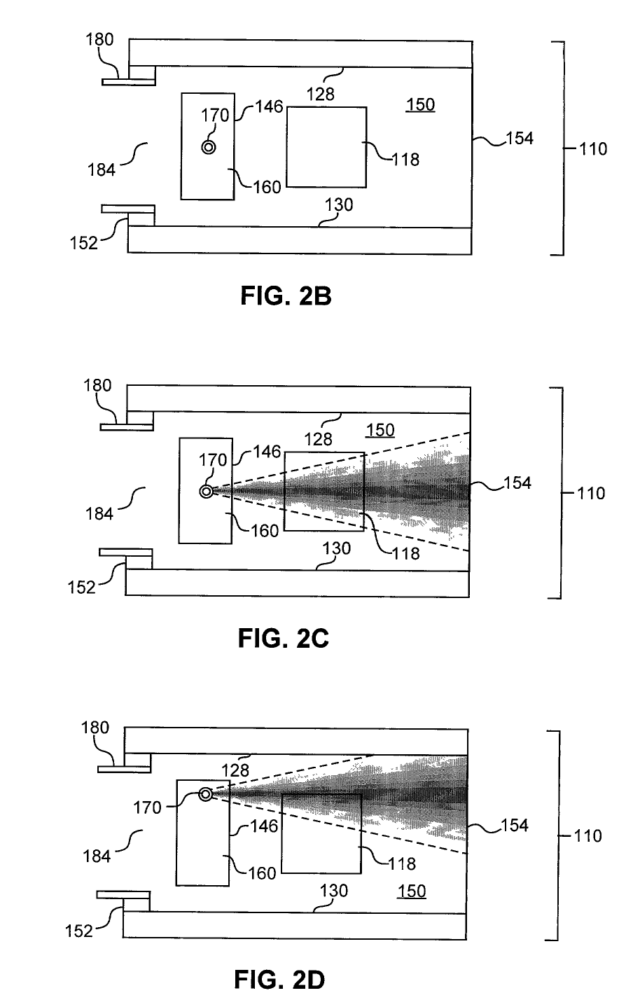

FIG. 2 illustrates a device that may be employed to carry out the inventive method to create a concentration gradient over the target region in the above-described manner. A similar device is described in U.S. Ser. No. ______ (“Method for Conducting Cell-Based Analyses Using Laminar Flow, and Device Therefor”) inventor David Socks, filed evendate herewith. Aspects of the flow passage design described therein may be employed in the present invention as well. As illustrated in FIG. 2A, device 110 includes a substrate 112 comprising first and second substantially planar opposing surfaces indicated at 114 and 116, respectively. The substrate 112 represents at least a portion of a flow passage 150 in which a concentration gradient of a reagent will form. A square-shaped target region 118 is located at the center of surface 114. In order to reduce the volume of reagent or the quantity of cells needed for chemotactic assays, the surface area of the target region is typically 1 mm² to about 100 mm², preferably about 10 mm² to about 50 mm², and optimally about 20 mm² to about 30 mm².

Also shown in FIG. 2A are an optional base 120 and an optional cover plate 140 that further define the flow passage. The base 120 has a channel 126 located on a surface 122, the channel defined by parallel opposing side walls 128 and 130 and floor 132, extending along the length of the base 120 from a carrier inlet terminus 134 and an outlet terminus 136. The channel is sized and shaped to snugly contain the substrate 112 such that fluid-tight contact can be established between side walls 128 and 130 and the substrate 112. The cover plate 140 is complementarily shaped with respect to the base 120. Contact surface 142 of the cover plate 140 is capable of interfacing closely with the contact surface 122 of the base 120 to achieve fluid-tight contact between the surfaces. The cover plate contact surface 142 in combination with the upper surface 114 of the substrate and the side walls 128 and 130 of the channel 126 define a flow passage 150 through which a carrier fluid may flow. As illustrated in FIG. 2B, carrier fluid opening 152 is located at the upstream end of the flow passage, and outlet 154 is located at the downstream end of the flow passage. When the contact surfaces of the cover plate and the substrate are in fluid-tight contact, the flow passage is fluid-tight as well.

An opening 146 located between the carrier flow inlet 152 and the target region 118 extends through the cover plate 140. Extending through an elastic septum 160 in fluid tight contact with the opening is an introduction tube 162 having an end in communication with the flow passage, the end representing an inlet 170 for introducing a reagent into the flow passage. An additional septum 180 is also provided in fluid-tight contact with the flow passage 150 at the carrier fluid opening 152 to allow carrier fluid tube 182 to convey carrier fluid through carrier fluid inlet 184 and into the flow passage 150.

As illustrated in FIG. 2B, the device is assembled to form a flow passage 150 defined by the substrate in the base, the side walls 128 and 130, and the cover plate. When the device is used for a cellular assay, cells may be immobilized on the target region before, during, or after device assembly. Once the device is assembled, a carrier fluid is introduced through the carrier fluid inlet 184 and maintained in contiguous laminar flow through the flow passage 150, over the target region 118, and through the outlet. As a result, the carrier fluid fills the entire flow passage 150 and flows over and covers the entire target region 118. Then, as illustrated in FIG. 2C, a stream containing a reagent is introduced through inlet 170, the introduction tube 162, and into the carrier fluid while the carrier fluid is still flowing through the flow passage 150. The stream is introduced as a laminar flow. As a result, the carrier fluid conveys the stream of reagent toward the outlet 154 of the device. However, both the flow of the carrier fluid and the reagent stream are sufficiently slow to allow the reagent that has entered the carrier fluid to diffuse in a direction perpendicular to the flow passage 150. Thus, as shown, the fluid that passes over a portion of the target region 118 exhibits a reagent concentration gradient. By carefully controlling the absolute and the relative flow rates of the carrier fluid and the reagent stream, the concentration gradient formed over the target region may be controlled as well. It should be noted that, while the inlet is shown located approximately equidistant from each of the side walls, the inlet may be located anywhere upstream from the target region as long as a desired concentration gradient is formed. Thus, for example, FIG. 2D illustrates that the inlet 170 may be positioned closer to side wall 128 than to side wall 130 if it is desired that the reagent concentration gradient in the target region is such that the reagent concentration decreases from side wall 128 to side wall 130.

This method provides a number of advantages over other methods in carrying out chemotactic assays. For example, by selecting a carrier fluid capable of sustaining living cells, the carrier flow may continuously deliver essential nutrients to the cells while flushing away nutrient-depleted and waste-filled carrier fluid. The method may be carried out to controllably produce static as well as dynamic concentration gradients over the cells through control of the flow of the carrier fluid and the reagent stream.

Another technique for effecting controlled delivery of a fluid containing a reagent involves introducing a plurality of fluids, each fluid introduced through an inlet to form a lane downstream from each inlet in contiguous laminar flow through the flow passage. At least one fluid contains the reagent. The reagent in the fluid is allowed to diffuse across one or more lanes to form a concentration gradient over the target region. This can be carried out by employing a device similar to the device illustrated in FIG. 2, except that a plurality of inlets is provided as a row wherein adjacent reagent streams of appropriate reagent concentrations are introduced into the carrier fluid. The reagent is then allowed to diffuse to form a desired concentration gradient profile over the target region.

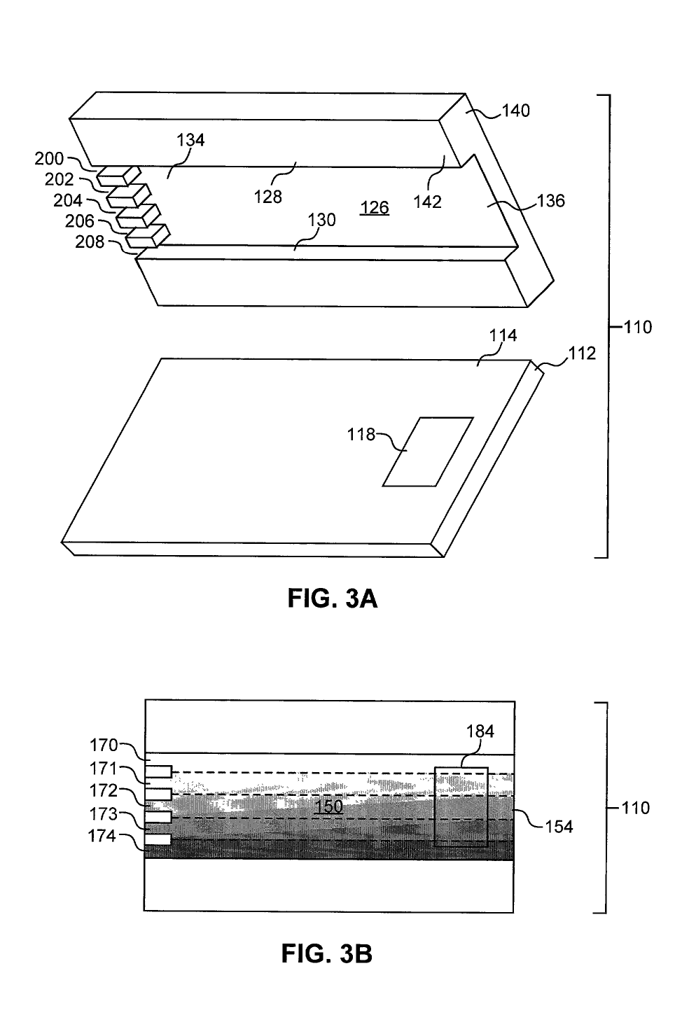

FIG. 3 illustrates another example of a device that may be employed to carry out the above-described method using a plurality of fluids. A similar device is described in U.S. Ser. No. ______ (“Method for Conducting Cell-Based Analyses Using Laminar Flow, and Device Therefor ”) by inventor David Socks, filed evendate herewith, as well. As illustrated in FIG. 3A, the device 110 includes a substrate 112 having a target region 118 located on surface 114. The device 110 also includes an optional cover plate 140 having a main channel 126 located on the first surface 142, as defined by opposing side walls 128 and 130 and ceiling 132 extending along the length of the cover plate 120. The main channel 126 has an inlet terminus 134 at a first end and an outlet terminus 136 at the opposing end. As shown in FIG. 3A, terminus 134 is located away from the exterior edges of the first cover plate surface 142, whereas terminus 136 is located at an edge of the first cover plate surface 142. A plurality of parallel introduction channels, in order indicated at 200, 202, 204, 206, and 208, extends from the exterior edge opposing the main channel outlet terminus 136, to the inlet terminus 134.

The contact surface 142 of the cover plate 140 is typically capable of interfacing closely with the contact surface 114 of the substrate 112 to achieve fluid-tight contact between the surfaces. The substrate contact surface 114 in combination with the ceiling 132 and the side walls 128 and 130 of the channel 126 define a main flow passage 150 through which fluids may flow. Similarly, the substrate contact surface 114 in combination with introduction channels 200, 202, 204, 206, and 208 form introduction conduits each having an inlet indicated at 170, 171, 172, 173, and 174 through which fluid external to the microdevice may flow, emptying into the main flow passage 150. Outlet 154 is located at the downstream end of the flow passage. The introduction conduits are typically provided fluid communication with a plurality of fluid sources.

In operation, as illustrated in FIG. 3B, the device is assembled to form the main flow passage 150 defined by the substrate, the side walls 128 and 130, and the ceiling of the main channel. The target region 118 is located within the main flow passage 150 downstream from the introduction conduits and associated inlets 170, 171, 172, 173, and 174. As shown, the inlets are positioned in a line perpendicular to the flow passage and parallel to the substrate surface. Each inlet is provided fluid communication with a fluid source. Fluid flows from the sources making it possible to maintain each of the fluids in contiguous laminar flow through the flow passage to form fluid lanes, whose boundaries are indicated by dotted lines, extending from each of the inlets 170, 171, 172, 173, and 174 over a portion of the target region 118. However the fluids in the lanes move at a sufficiently slow rate to allow diffusion of the reagent across the boundaries of the lanes. Thus, for example, inlets 170, 171, 172, 173, and 174 each provide a fluid of increasing reagent concentration. For example, fluid from inlet 170 may contain no reagent, fluid from inlet 174 may contain a high concentration of the reagent, and inlets 171, 172, and 173 may contain intermediate concentrations of the reagent as the fluids are introduced into the flow passage. Since these lanes may be quite narrow, a relatively short amount of time may be sufficient for the reagent to diffuse across the lanes to form a controlled gradient over the target region.

Similar to the method illustrated in FIG. 2, the formed concentration gradient may be static or dynamic. When the formed concentration gradient is dynamic, the concentration of the reagent in at least one fluid is altered during practice of the technique. Thus, this technique provides all of the advantages of the technique illustrated in FIG. 2 compared to gradient formation methods that do not employ laminar flow, and also provides additional advantages relating to speed of gradient formation and to gradient range. Because a plurality of fluids is employed, this method provides greater control over the concentration of a reagent on a particular location of the target region. In addition, even when employed to create a gradient that may also be created using the method illustrated in FIG. 2, the introduction of a plurality of fluids allows for reagent contained therein to diffuse over a shorter distance, thereby providing more rapid gradient formation.

For any of the above embodiments, at least one inlet is required for each reagent used in conjunction with the device. When a plurality of reagents is employed, the inlets may be positioned such that lanes of reagents do not contact each other, to allow for a gradient to be formed over a distinct region of the target surface to prevent reagent interaction. Alternatively, when interaction between reagent gradients is desired, the inlets may be positioned such that lanes of reagents contact each other, to allow for gradients of different reagents to be formed over the same region of the target surface.

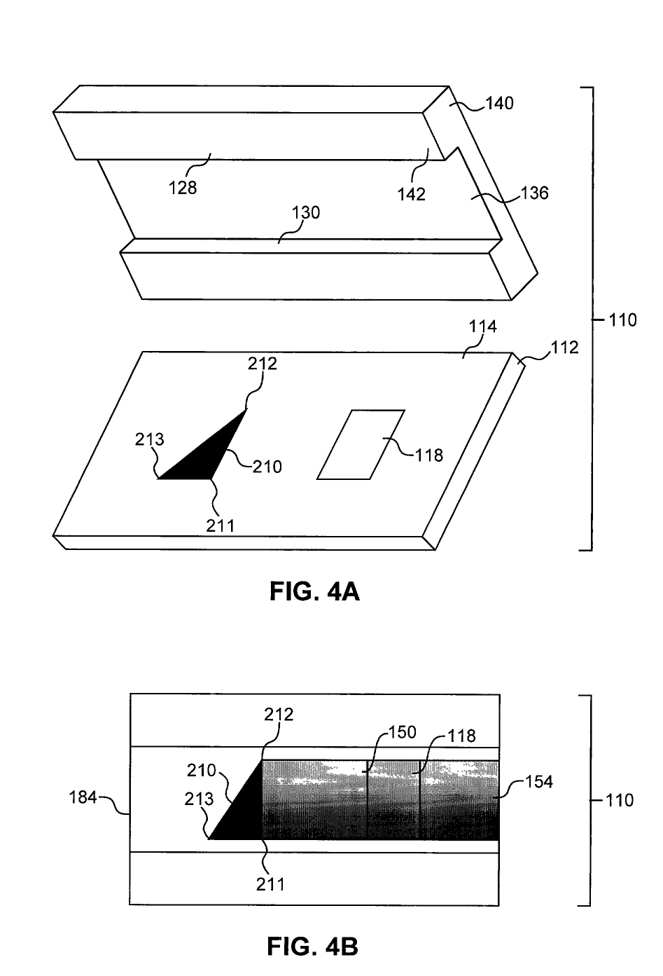

Still another technique for effecting controlled delivery of a fluid containing a reagent involves providing a reagent source located upstream from the target region. The reagent source is adapted to release reagent into the carrier fluid as the carrier fluid flows over the source of reagent. Thus, when the carrier fluid is maintained in contiguous laminar flow to contact the reagent source, reagent is released into the carrier fluid to form a concentration gradient downstream from the source and over the target region. As illustrated in FIG. 4, a device similar to that illustrated in FIG. 3 may be employed in this technique. Instead, no introduction channel (such as those indicated at 200, 202, 204, 206, and 208 in FIG. 3) and only one inlet 184 is needed. In addition, a reagent source 120 is provided on the substrate surface 114. As shown, the source 210 is a solid uniform layer affixed to the substrate surface and adapted to release reagent into a carrier fluid as the carrier fluid contacts the source 210. In addition, the layer is provided in the shape of a right triangle defined by vertices 211, 212, and 213, wherein vertex 211 represents the right angle vertex of the right triangle. The leg of the triangle extending between vertices 211 and 213 is substantially parallel and congruent with a side of the target region.

As illustrated in FIG. 4B, once the device is assembled to form a flow passage, a carrier fluid is introduced through the carrier fluid inlet 184 and maintained in contiguous laminar flow through the flow passage 150, over the reagent source and the target region 118, and through the outlet. As a result, the carrier fluid fills the entire flow passage 150, contacts the reagent source, and flows over and covers the entire target region 118. As the carrier fluid contacts the reagent source, the reagent source releases reagent into the carrier stream. As a result, the carrier fluid conveys the reagent over the target region toward the outlet 154 of the device. Since fluid that flows over the reagent source near vertex 212 contacts the source 210 for a shorter time period than fluid that flows over the source near vertex 211, fluid downstream from vertex 212 exhibits a lower concentration of the reagent than the fluid downstream from vertex 211. Thus, a reagent concentration gradient is formed over the target region.

When the reagent is a solid, the layer may consist essentially of the reagent as a coating, a pressed pellet, or other solid form. Alternatively, solid or non-solid reagents may be compounded with an additional material that serves as a binder to form a matrix adapted to controllably release reagent into a carrier upon contact. In such a case, the binder material may swell or be solvated by the carrier to release the reagent into the carrier fluid. When the carrier fluid is aqueous, the binder material may be collagenic or another type of hydrophilic substance such as a hydrophilic polymer. Suitable hydrophilic polymers include, for example, polyalkyleneoxides such as, for example, PEG and polypropylene glycol (PPG), polyvinylpyrrolidones, polyvinylmethylethers, polyacrylamides, such as, for example, polymethacrylamides, polydimethylacrylamides and polyhydroxypropylmethacrylamides, polyhydroxyethyl acrylates, polyhydroxypropyl methacrylates, polymethyloxazolines, polyethyloxazolines, polyhydroxyethyloxazolines, polyhyhydroxypropyloxazolines, polyvinyl alcohols, polyphosphazenes, poly(hydroxyalkylcarboxylic acids), polyoxazolidines, polyaspartamide, polymers of sialic acid (polysialics), copolymers thereof, and mixtures thereof. Such hydrophilic materials may be additionally compounded with a hydrophobic material such as a wax or petroleum jelly to slow the release of the reagent in contact with an aqueous carrier.

The reagent and the binder material may be provided in an appropriate ratio to release the reagent at a constant rate. When the binder material is polymeric, such as one listed supra, the molecular weight of the binder polymer may be selected according to the desired reagent release rate. Typically, higher molecular weight polymers will result in a slower release rate. In addition, it is preferred that the binder material be substantially immobile with respect to the substrate, to avoid release of the binder material downstream if the binder material will interfere with the function of the reagent or concentration gradient formation over the target region. For example, if the method is employed to carry out chemotactic assays and the binder material has a potential to interfere with the results of the chemotactic assay, it is preferred that the binder material not be released into the fluid. Thus, the binder material may be covalently bound to the substrate surface. In certain chemotactic assays, it may be appropriate to employ the same material as a binder for the reagent source and to immobilize the cells. That is, for example, if a collagenic material may be employed to immobilize cells on a target region, the same collagenic material may be employed as a binder that assists in controlling the release of the reagent into the carrier fluid flowing through the flow passage.

One skilled in the art will recognize that since contact time between carrier fluid and the source will greatly affect the reagent concentration downstream, the reagent source may be shaped appropriately to create the desired concentration gradient. Thus, depending on the desired concentration gradient, the source may take non-triangular forms as well. For example, if it is desired that only half of the target region be exposed to reagent, then the source may be shaped such that at least half of the target region is not located downstream from the source.

Thus, this technique provides a number of the advantages of the techniques illustrated in FIGS. 2 and 3 with respect to gradient formation. For example, this technique provides gradient formation control as a function of carrier fluid flow as well as the properties and characteristics of the reagent source. In addition, this method does not rely on the reagent diffusion between fluid lanes in gradient formation.

A further technique for effecting controlled delivery of a fluid containing a reagent involves sweeping a hydrodynamically focused stream of fluid over the target region while simultaneously ensuring correspondence of the concentration of the reagent in the stream to a predetermined concentration profile. Use of hydrodynamic focused streams in cellular assays has been described, for example, in U.S. Ser. No. ______ (“Flow Cell Assemblies and Methods of Spatially Directed Interaction Between Liquids and Solid Surfaces”), inventors Martin Bonde and Thomas Ahl, filed on Jun. 29, 2001; aspects of hydrodynamic focusing described in this application may be employed in the present invention as well.

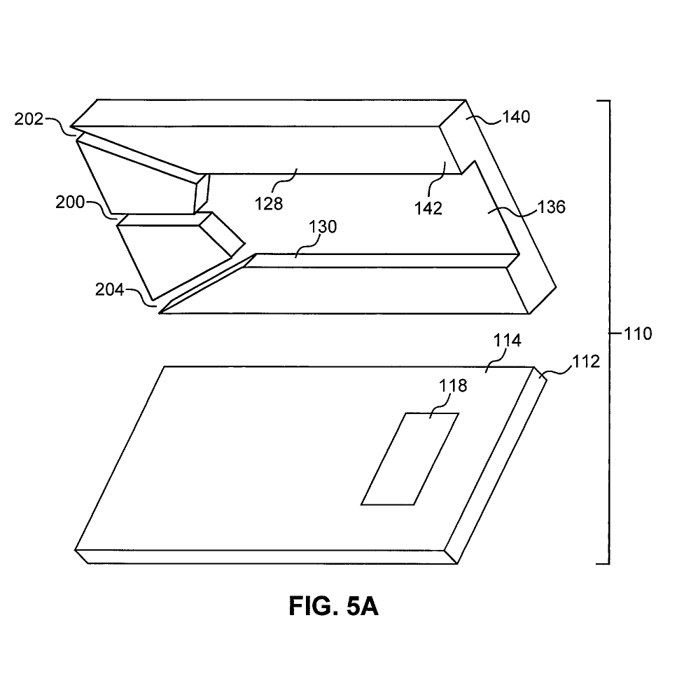

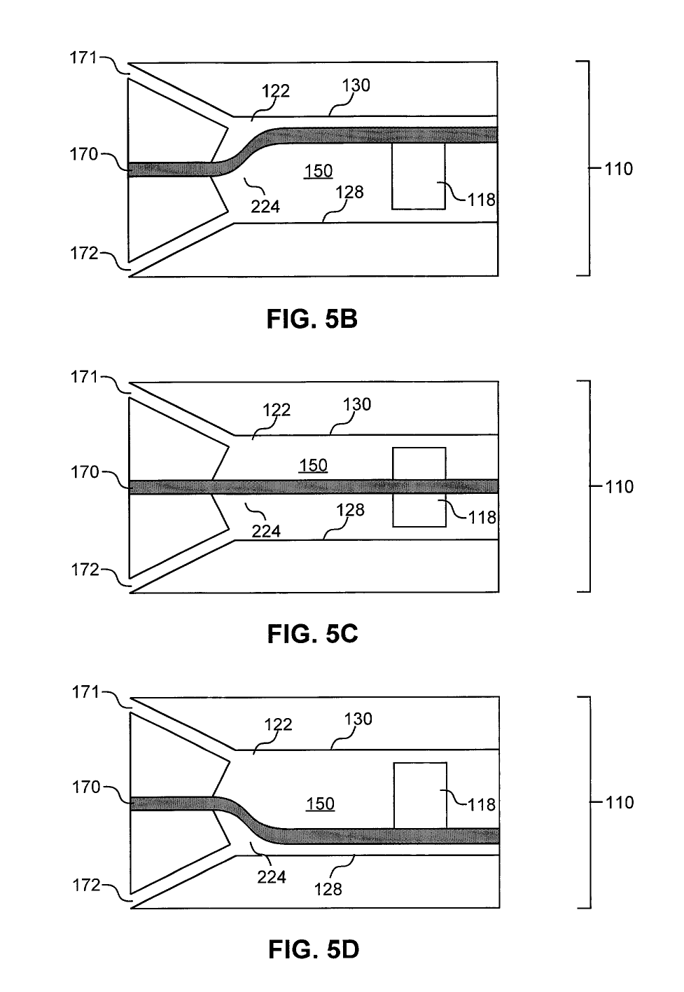

FIG. 5 illustrates a device that may be employed to sweep a hydrodynamically focused stream over the target region. As illustrated in FIG. 5, a device similar to that illustrated in FIG. 3 may be employed in this technique. However, three introduction channels are provided. That is, a reagent stream channel 200 is provided between two guide stream channels, indicated at 202 and 204 on the cover plate contact surface, such that when the cover plate contact surface is placed in contact with substrate surface 114, channels 200, 202 and 204 form introduction conduits each having an inlet indicated at 170, 171, and 172 through which fluid external to the microdevice may flow, emptying into the main flow passage 150. As shown, guide stream inlets 171 and 172 are located at the most upstream position on sidewalls 128 and 130, respectively.

In operation, as illustrated in FIGS. 5B, 5C, and 5D, the device is assembled to form the main flow passage 150 defined by the substrate, the side walls 128 and 130, and the ceiling of the main channel. The target region 118 is located within the main flow passage 150 downstream from the introduction conduits and associated inlets 170, 171, and 172. The guide stream inlets are provided fluid communication with a guide fluid source and the reagent inlet is provided fluid communication with a fluid source containing the reagent. When fluid flow is provided from the sources and through inlets 170, 171, and 172, a lane of reagent 220 is formed between two lanes 222 and 224 of guide fluids. Generally, the width of the reagent lane is a function of the volumetric flow rate of the fluid in the reagent lane and the flow rate of the guide streams. That is, a low reagent fluid flow rate in combination with a high guide stream flow rate tends to result in a narrow reagent lane. Conversely, a high reagent fluid flow rate in combination with a low guide stream flow rate tends to result in a wide reagent lane. Typically, the lane width is equal to or smaller than the average cell diameter in order to carry out chemotaxis.

In addition, the position of the reagent lane depends on the relative flow rate of the fluids in the guide lane. For example, FIG. 5B illustrates the position of the reagent lane when the volumetric flow rate of the fluid in lane 224 is substantially greater than that of the fluid in lane 222. FIG. 5C illustrates the position of the reagent lane when the volumetric flow rates of the fluids in lanes 222 and 224 are approximately equal. FIG. 5D illustrates the position of the reagent lane when the volumetric flow rate of the fluid in lane 224 is substantially lower than that of the fluid in lane 222. It should be evident, then, that it is possible to sweep the hydrodynamically focused reagent stream 220 from side wall 130 to side wall 128 over the target region 118 by increasing the flow rate of fluid in lane 222 to the flow rate of fluid in lane 224. The combined volumetric flow rate of the fluids in lane 222 and lane 224 should remain constant to maintain the width of the reagent stream 220. Similarly, a hydrodynamically focused stream of reagent may be swept from side wall 128 to side wall 130 over the target region 118 by increasing the flow rate of fluid in lane 224 to the flow rate of fluid in lane 222. Such sweeping may be repeated in the same direction or in alternating directions. In addition, the reagent stream 220 may be swept over the entire target region 118 or a portion thereof.

While the hydrodynamically focused stream of reagent is swept over the target region, the concentration of the reagent in the stream is simultaneously altered. This alteration exposes the target region to a dynamic gradient. The reagent concentration alteration may involve increasing or decreasing the reagent concentration over time. Thus, the reagent concentration alteration may result in the flow of a stream of fluid that exhibits a predetermined concentration profile of the reagent.

This method provides a number of advantages over other methods in carrying out chemotactic assays. For example, by selecting a reagent or guide stream fluid capable of sustaining living cells, the carrier flow may continuously deliver essential nutrients to the cells while flushing away nutrient-depleted and waste-filled carrier fluid. The method may be carried out to controllably produce dynamic concentration gradients over the cells through control of the flow of the guide and reagent lane fluids.

Although it is a straightforward matter to produce an ordinary stream of fluid that exhibits a controllably varying concentration of reagent, this is not the case with streams that contain extremely small volumes of fluid due the difficulty in achieving proper mixing. For example, an ordinary stream of fluid that exhibits a controllably varying concentration of reagent may be produced simply by controlling the flow rates of two streams of different reagent concentrations that are mixed to form one stream. Mixing generally involves turbulent fluid flow, which is difficult to achieve with small volumes of fluid. In addition, commercially available mixing apparatuses generally require a minimum volume of fluid in order to operate.

Thus, another embodiment of the present invention relates to a method for producing a stream of fluid having a predetermined concentration profile of a reagent. This inventive method may be employed to produce a stream of fluid exhibiting a controllably varying concentration of reagent in a small volume. The method involves providing a fluid vessel having a cavity extending from an inlet opening to an outlet opening and loading a plurality of fluids, each fluid containing a different concentration of the reagent in a sequence though the inlet opening into the cavity. The sequence is selected to correspond to a predetermined concentration profile of the reagent. The loaded fluid is expelled through the outlet opening and out of the vessel to produce a stream of fluid that exhibits the predetermined concentration profile of the reagent. For small volumes of fluid, the fluid vessel may be a capillary tube. The predetermined reagent concentration profile may exhibit an increasing or decreasing reagent concentration. Sufficient time may be allowed to permit the reagent to diffuse within the cavity before expelling the loaded fluid, if the predetermined concentration profile to be formed is gradual. Optimally, the vessel contains no discontinuities in fluid, e.g., no bubbles, before the loaded fluid is expelled.

The sequential loading of the vessel with fluid of varying reagent concentrations may be carried out using manual or automated fluid handling devices. For example, the wells in microtiter well plates having 96, 384 or 1536 wells may each contain a fluid of a different reagent concentration. A quantity of fluid may be withdrawn from each well and loaded in sequence into the inlet opening of a capillary tube to result in the formation of the predetermined concentration profile. Once sufficient time has passed to allow reagent diffusion to take place, pressure is applied to the inlet opening through any of a number of pressure generating means, e.g., syringe, micropump, etc., to eject a stream of fluid exhibiting the desired concentration profile out of the outlet opening of the capillary.

It should be noted that when a hydrodynamically focused stream of fluid is swept over the target region, a constant reagent concentration may be used in the stream to create a “time-modulated” gradient zone on the target area. In this approach, the stream of constant reagent concentration is displaced to create a fluctuation or oscillation in lane position. This may involve quickly varying the flow rate of the guide streams and/or the reagent stream. These short-term lane fluctuations result in the creation of a time-modulated concentration around the “average lane” position. As a result, the portion of the target region at the average lane position will be exposed to more reagent than portions of the target region away from the average lane position. For example, since a typical reagent stream may have a width of about 10 to about 25 micrometers, many chemotaxis experiments can be conducted on a 5 mm wide target region having cells immobilized thereon. Moreover, this approach can be employed wherein a plurality of lanes (ranging from two to eight or more) is employed. In such a case, a time-modulated gradient may be generated at each lane or at specific selected lanes. Because time-modulated gradient generation typically involve contact fluctuations between the reagent and the target region (and cells immobilized thereon) in the millisecond range, the time-modulated gradient is particularly useful in studying the kinetic binding behavior of reagents to cell receptors with low affinities.

With respect to gradient formation control using laminar flow, the geometry of the device components plays an important role in determining the control over the accuracy and precision of the fluid flow. Thus, while the substrate is the only necessary component that provides a surface that defines the flow passage, it is preferred that the flow passage is further defined by other components as well. As discussed above, the flow passage is typically defined in part by a cover plate that opposes the target region of the substrate surface as well. Often, the cover plate surface is parallel to the target region of the substrate surface. Similarly, it is preferred that the flow passage of the device is constructed as a conduit. Accordingly, the flow passage is typically defined by opposing side walls in fluid-tight contact with the substrate as well. In some instances, the side walls represent an integral portion of the substrate. When the flow passage is a conduit having a constant cross-sectional shape and area, formed lanes are substantially parallel to each other and to the conduit walls. This arrangement is preferred for the formation of a linear concentration gradient. Conversely, the width of the lanes may vary according to variations of the cross-sectional shape and area of the conduit. One skilled in the art will recognize that lanes may be narrowed if the conduit is narrowed as well.

Similarly, the cover plate and substrate surfaces may or may not be parallel to each other. As reagents and fluids that may be employed with the invention may be rare or expensive, it is preferred that as little reagent and fluid is used to flow over the target region as is practicable. However, fluid flow depends on the volume of reagent or fluid as well as the volume of the flow passage. Typically, when the substrate and cover plate surfaces are parallel to each other, the surfaces are located from about 1 μm to about 500 μm from each other. Preferably, the substrate and cover plate surfaces are located from about 20 μm to about 100 μm from each other.

For any of the embodiments described above, it is preferred that the device be constructed in a modular manner to ensure the interchangeability of the components. In particular, certain components may be formed from stock items to lower the cost of the device and to make it cost effective to treat at least stock components as disposable. For example, the substrate may consist of an ordinary 25 mm×75 mm or 50 mm×75 mm glass slide found in most laboratories. Similarly, to facilitate handling, the components of the inventive device may be detachable from each other. As access to the target region of the substrate is limited when it is in opposing relationship to the cover plate, it is preferred that substrate be detachable from the cover plate. When the substrate is a detachable and disposable item such as glass slide, complex capillary tube attachment procedures may be avoided before each use of the device when the tubes are essentially permanently connected to the inlets.

The device may be adapted to form reagent gradients from fluids of time-modulatedly any type and amount desired depending on the intended purpose of gradient formation. Thus, the fluid may be aqueous and/nor nonaqueous. Nonaqueous fluids include, for example, organic solvents and lipidic liquids.

Since fluid laminar flow is a function of a number of variables such as the geometry of the surfaces over which the fluid flows, flow velocity, and fluid properties such as viscosity, it is important that fluid movement in the inventive device be precisely controlled. Inlets through which fluids containing reagents are introduced into the flow passage typically have a cross sectional area of 1×10⁻⁵ mm² to about 1 mm², preferably about 5×10⁻⁴ to about 0.1 mm², and optimally 1×10⁻³ mm² to about 1×10⁻² mm². The inlets may have a variety of shapes including, but not limited to, circular, elliptical, square, rectangular, and triangular.

In order to ensure that laminar flow is exhibited in the fluid flowing through the flow passage, a pump is employed to delivery appropriate fluid from a fluid source through the appropriate inlet. Typically, high precision microsyringe pumps are employed to provide fluid flow through capillaries to the inlets. However, other types of pumps may be employed as well. In some instances, one pump is sufficient to provide a motive force to ensure proper fluid flow. For time-modulated gradient generation as described above, a means for creating pressure fluctuations may be employed. Such means may involve piezoelectric or other known vibration producing elements as well.

It should be noted that a fluid exhibiting laminar flow containing a concentration gradient of a reagent over the target region may be employed to form an immobilized reagent layer. That is, fluid flowing over the target region delivers reagent to the target region, and the reagent, as a result, is immobilized as a reagent layer to the target region. Immobilization may occur as the result of chemical attachment through covalent, ionic or other bonds. The reagent is attached preferentially to portions of the target region that are exposed to fluid containing a high reagent concentration than to portions that are exposed to fluid containing a low reagent concentration. For example, a number of surface reactive sites on the target region may be provided to react with the reagent. As reagent in the fluid is delivered according to the concentration gradient in the fluid, reagent is likewise attached to the target region. As a result, the reagent gradient is “captured” or “imaged” on the target region. That is, the attached reagent layer exhibits a reagent layer concentration gradient that corresponds to the concentration gradient of fluid flowing over the target region. Optionally, attachment between the surface reactive sites and the reagent is covalent in nature.

The inventive devices described herein can be adapted for use in connection with a cell-based assay, particularly a chemotactic assay. Thus, the invention also relates to a method for carrying out a cellular assay. As before, flow passage is provided that is defined at least in part by a substrate. However, a cell is immobilized on a surface of the substrate. A concentration gradient of a reagent is formed over the cell by controlled delivery of a fluid containing the reagent in laminar flow through the flow passage and over the target region. The concentration gradient may be formed through any of the techniques discussed above. Alternatively, the concentration gradient may be attached to the target region as discussed above. After the cell is exposed to the concentration gradient, the method involves detecting a cellular response to the concentration gradient. Typically, the detection step involves detecting chemotaxis. This may involve detecting for a change in the position of a portion of the cell such as the nucleus or the entire cell. In the alternative or in addition, the detection step may involve detecting a change in the shape of the cell. Furthermore, a number of indirect methods may also be employed for detecting cell movement. For example, when a cell is immobilized on a surface, cell movement may be detected based on the disruption of the surface on which the cell immobilized. In some instances, fluorescent beads may be interposed between the surface and the cell. As the cells move, the beads are phagocytized. As a result, the cellular movement causes the clearing of a path of non-fluorescing material. In other instances, specific probes may be used as antibodies to determine the morphology of intracellular cytoskeleton proteins. In such an instances, rearrangement of intermediate filaments and microtubules indicates cell movement.

Such a method can be carried out by adapting any of the above devices to include any detector known in the art for detecting a cellular response to the concentration gradient, e.g., moving, changing shape, or expressing a protein. For example, the detector may comprise an optical imaging system or a microscope for detecting movement of any portion of an immobilized cell. Other detectors include, for example, chromatographic detectors, an immunoassay, a fluorescence detector, a radioactivity detector, and combinations thereof.

When cells are immobilized on a substrate surface, it is preferred that fluid flowing over immobilized cells comprise a culture medium containing nutrients for sustaining the viability of the cell in addition to providing directionality to the stream of fluid containing the reagent. It must be noted, however, that the culture medium does not necessarily ensure that the cell remains living, although living cells are preferred. Thus, for example, the culture medium may be provided to keep living cells viable in the absence of a toxic reagent. If a toxic reagent is introduced into the flow cell, e.g., during a toxicity study, cell death may result notwithstanding the presence of the culture medium. Of course, the inventive methods and devices also provide a convenient means for evaluating toxicity as a function of reagent concentration.

Culture media suitable for any particular cell will be known to those skilled in the art and are available commercially from, for example, Sigma Inc., St. Louis, Mo. Generally such media contain mixtures of salts, amino acids, vitamins, nutrients, and other substances necessary to maintain cell health. Preferred salts in the culture medium include, without limitation, NaCl, KCl, NaH₂PO₄, NaHCO₃, CaCl₂, MgCl₂, and combinations thereof. Preferred amino acids are the naturally occurring L amino acids, particularly arginine, cysteine, glutamine, histidine, isoleucine, leucine, lysine, methionine, phenylalanine, threonine, tryptophan, tyrosine, valine, and combinations thereof. Preferred vitamins in the cell culture include, for example, biotin, choline, folate, nicotinamide, pantothenate, pyridoxal, thiamine, riboflavin, and combinations thereof. Glucose and/or serum, e.g., horse serum or calf serum, are also preferred components of the culture medium. Optionally, antibiotic agents such as penicillin and streptomycin may be added to suppress the growth of bacteria. Preferably, the culture medium will contain one or more protein growth factors specific for a particular cell type. For example, many nerve cells require trace amounts of nerve growth factor (NGF) to sustain their viability. Similarly, the culture medium will preferably contain hepatocyte growth factor (HGF) when hepatocytes are present in the assay. Those skilled in the art routinely consider these and other factors in determining a suitable culture medium for any given cell type. The culture medium can be present in one or both of the guide streams and optionally in the fluid stream containing the reagent.

Nearly any type of cells may be used with the present methods, including both eukaryotic cells and prokaryotic cells. Preferably, however, the cell is a primary cell obtained from a mammal, e.g., a human. Preferred cell types are selected from the group consisting of blood cells, stem cells, endothelial cells, epithelial cells, bone cells, liver cells, smooth muscle cells, striated muscle cells, cardiac muscle cells, gastrointestinal cells, nerve cells, and cancer cells. Alternatively, the immobilized cells may originate from a cell line.

The substrate surface on which the target region is located may be selected for facile immobilization of cells. Such solid surfaces include, for example, a collagen-derivatized surface, dextran, polyacrylamide, nylon, polystyrene, and combinations thereof. Typically, immobilized cells are present on the target region as a monolayer. The monolayer may be substantially contiguous or comprise an array of features, each feature comprising at least one cell. All or substantially all of the immobilized cells may be of the same type. Alternatively, the immobilized cells may be different, e.g., from two distinct cell lines. The monolayer may be immobilized on the solid surface using conventional techniques known to those skilled in the art. For example, the cells may be immobilized on the target region by simply contacting the target region with the cells. Optionally, a centrifuge may be used. Generally, the force required to immobilize the cell on the target region is from about 200× g to about 500× g. Further optionally, the cells are delivered to the target region through laminar flow, that is, one or more cells may be placed and immobilized on a target region through use of laminar flow cellular delivery technologies known in the art.

Alternatively, the surface may be coated with a coating of cell-adhereing substance such as collagen, alginate, agar, or other material to immobilize the cells. When immobilization of cells in a contiguous layer is desired, the cell-adhering substance may be contiguously coated on the target region. However, when it is desirable to provide an immobilized array of cells, the cell-adhering substance may be present as an array of features on the target region. That is, an array of locations on the target region may be coated with an appropriate material to form an array, e.g., lanes, checkerboard, spots or other pattern, so that cells may be spatially arranged at specific locations on the solid surface. See, e.g., U.S. Pat. Nos. 5,976,826 and 5,776,748 to Singhvi. In some instances, a photolithographic technique may be employed. U.S. Pat. Nos. 5,202,227 and 5,593,814 each to Matsuda et al. describe a process for preparing a cell arrangement control device wherein a photosensitive, cell-nonadhesive polymer is applied to a cell adhesive surface. The resulting photosensitive cell-nonadhesive polymer layer is irradiated patternwise and developed to leave the irradiated portion on the cell adhesive surface, thereby providing a pattern of the cell-nonadhesive polymer on the cell adhesive surface. As a result, a biological cell culture device may include a surface pattern having a cell adhesive portion and a cell-nonadhesive portion, wherein the cell-nonadhesive portion is covalently bound to the cell adhesive surface.

When a reagent gradient is attached to the target region, chemotactic evaluation may be carried out by immobilizing cells over the attached reagent gradient on the target region. In some instances, the attached reagent gradient may serve to immobilize the cells. Typically, cells are immobilized over only a portion of the gradient, e.g., the portion that contains a lower reagent concentration. In such a case, the chemotactic properties of the gradient may be evaluated by determining the movement of the immobilized cells in response to the attached gradient.

Alternatively, the cells are present on the target region as a tissue sample. Immobilization of tissue samples containing cells of interest may be accomplished by first freezing, e.g., to about −15 ° C. to about −20 ° C., a relatively large section of tissue. Thereafter, a knife, microtome, or similar sectioning device is used to slice the frozen tissue into sections. Next, a single section of the tissue is placed onto the target region, e.g., a glass slide, and the section is allowed to “melt” on the target region, thereby immobilizing the cells in the tissue onto the target region. Those skilled in the art will recognize other immobilization techniques that can be used as well.

Once the cell or tissue containing the cells of interest is immobilized for chemotactic assays, the immobilized cells and/or portions thereof must be able to “sense” a reagent gradient and to move in response to the gradient. Individual immobilized cells, i.e., cells not from a tissue sample, should be sufficiently separated from each other for chemotactic evaluation. In any case, it is preferred that the gradient contacts the cell or cells of interest.

As will be appreciated, different assays require the detection of different types of biological activity. In some cases, determining a particular biological activity of a reagent can be accomplished by direct observation of the cell. For example, toxicity assays of a reagent involve detecting, for example, cellular death as a function of reagent concentration. In other assays, it is preferred to detect changes caused by the cell. For example, determining biological activity may be accomplished by assaying outflow material to detect substances excreted by the cell in response to the reagent concentration gradient. In addition, the detection may be carried out downstream from the target region from any of a number of directions as described in U.S. Ser. No. ______, (“A Method for Interacting a Product Substance with a Substance Retained on a Surface”) inventors Michael Beyer, Ulrich Krühne and Thomas Ahl, filed Apr. 25, 2001.

Thus, the cell-based assays described herein are useful for screening the effect of reagent (e.g., drug or drug candidate) concentration gradients on a number of biological activities. Examples of biological activities that can be screened include, without limitation, cellular differentiation, locomotion, toxicity, apoptosis, adhesion, translocation of signaling molecules, protein expression, induction or repression of signal transduction pathways, and oncogenic transformation. In addition, the present method allows for the ability to screen for adsorption, distribution, metabolism, and/or excretion properties of a reagent.

Variations of the present invention will be apparent to those of ordinary skill in the art. For example, while a channel may be provided on a cover plate or base surface, as described above, the channels may be instead located in the substrate surface. In addition, the inventive device may be employed to carry out biomolecular assays by immobilizing biomolecules in place of cells on the target region. Furthermore, the inventive device may be used in surface modification processes that involve the formation of gradient. For example, by selecting an appropriate etchant and substrate material, the target region of a substrate surface may be etched to exhibit different degrees of roughness in one procedure by proper use of an etchant concentration gradient. In addition, while several techniques have been described for the controlled formation of reagent concentration and reagent concentration gradients, these techniques are provided as examples only, and other techniques that incorporate a combination of different aspects of the techniques described herein may be employed as well.

It is to be understood that while the invention has been described in conjunction with the preferred specific embodiments thereof, that the foregoing description is intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

We claim:

1. A method for forming a concentration gradient of a reagent for use in a chemotactic evaluation, comprising:
(a) providing a flow passage defined at least in part by a substrate having a target region on a surface thereof; and
(b) forming a concentration gradient of a reagent over the target region by controlled delivery of a fluid containing the reagent in laminar flow through the flow passage and over the target region, wherein the concentration gradient formed is suitable for use in chemotactic evaluation.

2. The method of claim 1, wherein step (a) comprises placing the substrate surface in opposing relationship with a cover plate to further define the flow passage.

3. The method of claim 1, wherein step (a) comprises placing the substrate surface in fluid-tight contact relationship with opposing side walls to further define the flow passage.

4. The method of claim 1, further comprising, before step (b), (a′) immobilizing a cell on the target region.

5. The method of claim 4, wherein step (a′) comprises immobilizing a plurality of cells on the target region.

6. The method of claim 5, wherein the cells are sufficiently separated from each other for chemotactic evaluation.

7. The method of claim 5, wherein the plurality of cells is immobilized as a monolayer.

8. The method of claim 7, wherein the monolayer comprises an array of features, each feature comprising at least one cell.

9. The method of claim 5, wherein the plurality of cells is immobilized as a tissue sample.

10. The method of claim 5, wherein substantially all of the immobilized cells are the same type.

11. The method of claim 4, wherein the cell is a primary cell.

12. The method of claim 4, wherein the cell is from a cell line.

13. The method of claim 1, further comprising, during step (b):
(b′) allowing surface reactive sites on the target region to react with the reagent in the fluid to attach the reagent to the target region so as to form an attached reagent layer on the target surface that exhibits a reagent layer concentration gradient.

14. The method of claim 13, wherein the reaction between the surface reactive sites and the reagent results in covalent attachment between the surface reactive sites and the reagent.

15. The method of claim 13, further comprising, after step (b′), (b″) immobilizing a cell on the attached reagent layer.

16. The method of claim 15, wherein the attached reagent layer serves to immobilize the cell.

17. The method of claim 15, wherein the cell is placed and immobilized on the attached reagent layer through use of laminar flow cellular delivery.

18. The method of claim 15, wherein the cell is immobilized on the attached reagent layer according to the reagent layer concentration gradient.

19. The method of claim 1, wherein the reagent is contained in the fluid in solvated form.

20. The method of claim 1, wherein the reagent is contained in the fluid in partially solvated form.

21. The method of claim 1, wherein the reagent is contained in the fluid in suspended form.

22. The method of claim 1, wherein step (b) comprises:
(b′) introducing a plurality of fluids, each fluid introduced through an inlet to form a lane downstream from each inlet in contiguous laminar flow through the flow passage, wherein at least one fluid contains the reagent; and
(b″) allowing the reagent to diffuse across one or more lanes to form a concentration gradient suitable for chemotactic evaluation over the target region.

23. The method of claim 22, wherein at least one fluid contains no reagent before step (b″) is carried out.

24. The method of claim 22, wherein at least three lanes are formed in step (b′) and exhibit increasing concentrations of the reagent in a direction perpendicular to the flow passage and parallel to the substrate surface.

25. The method of claim 22, wherein the formed concentration gradient is static.

26. The method of claim 22, wherein the formed concentration gradient is dynamic.

27. The method of claim 26, wherein the concentration of the reagent in at least one fluid is altered during step (b′).

28. The method of claim 1, wherein step (b) comprises:
(b′) maintaining a carrier fluid in contiguous laminar flow at a selected flow rate through the flow passage and over the target region; and
(b″) introducing a stream containing a reagent through an inlet into the carrier fluid upstream from the target region at a flow rate appropriate to the selected flow rate of the carrier fluid to allow the reagent to diffuse downstream in the carrier fluid to form a concentration gradient over the target region.

29. The method of claim 28, wherein the carrier fluid is a medium appropriate to sustain living cells.

30. The method of claim 1, wherein step (b) comprises:
(b′) providing a source of reagent located upstream from the target region adapted to release reagent into the carrier fluid as the carrier fluid flows over the source of reagent; and
(b″) maintaining a carrier fluid in contiguous laminar flow at a selected flow rate through the flow passage to contact the source of reagent such that reagent is released into the carrier fluid to form a concentration gradient over the target region

31. The method of claim 1, wherein step (b) comprises (b′) sweeping a hydrodynamically focused stream of fluid over the target region while simultaneously ensuring correspondence of the concentration of the reagent in the stream to a predetermined concentration profile.

32. The method of claim 31, wherein the hydrodynamically focused stream of fluid is swept over the target region by adjusting the relative volumetric flow rates of guide streams that serve to focus the hydrodynamically focused stream.

33. The method of claim 32, wherein at least one guide stream contains a fluid selected to sustain a cell.

34. The method of claim 31, wherein step (b′) is repeated.

35. The method of claim 34, wherein step (b′) is repeated in the same direction.

36. The method of claim 34, wherein step (b′) is repeated at a rate sufficient to generate a time-modulated gradient.

37. The method of claim 31, wherein the concentration of the reagent in the stream is increased.

38. The method of claim 31, wherein the concentration of the reagent in the stream is decreased.

39. A device for forming a concentration gradient of a reagent for use in a chemotactic evaluation, comprising:
a flow passage defined at least in part by a substrate having a surface and a target region thereon; and
a means for forming a concentration gradient of a reagent over the target region by controlled delivery of a fluid containing the reagent in laminar flow through the flow passage,
wherein the concentration gradient is suitable for chemotactic evaluation.

40. The device of claim 39, wherein the means for forming a concentration gradient over a target region comprises:
a means for maintaining a carrier fluid in contiguous laminar flow at a carrier flow rate through the flow passage and over the target region;
an inlet in fluid communication with a source of a reagent and upstream from the target region; and
a means for delivering a stream of reagent through the inlet at a flow rate appropriate to allow the reagent to diffuse in the carrier to form a concentration gradient over the target region.

41. The device of claim 39, wherein the means for forming a concentration gradient over a target region comprises:
a plurality of inlets each located upstream from the target region; and
a means for introducing a plurality of fluids, each fluid introduced at a flow rate from a fluid source through an inlet to form a lane downstream therefrom exhibiting contiguous laminar flow through the flow passage, wherein each fluid contains a concentration of the reagent and the flow rates are selected to allow the reagent in the fluids to diffuse across one or more lanes to form a concentration gradient over the target region.

42. The device of claim 39, wherein the means for forming a concentration gradient over a target region comprises:
a means for maintaining a carrier fluid in contiguous laminar flow at a carrier flow rate through the flow passage and over the target region; and
a source of reagent located upstream from the target region and adapted to release reagent into the carrier fluid as the carrier fluid contacts the source of reagent such that a concentration gradient is formed over the target region.

43. The device of claim 39, wherein the means for forming a concentration gradient over a target region comprises
a means for sweeping a hydrodynamically focused stream of fluid over the target region while simultaneously ensuring correspondence of the concentration of the reagent in the stream to a predetermined concentration profile.

44. A method for producing a stream of fluid having a predetermined concentration profile of a reagent, comprising:
(a) providing a fluid vessel having a cavity extending from an inlet opening to an outlet opening;
(b) loading a plurality of fluids, each fluid containing a different concentration of the reagent in a sequence though the inlet opening into the cavity, wherein the sequence is selected to correspond to a predetermined concentration profile of the reagent; and
(c) expelling the loaded fluid through the outlet opening and out the vessel to produce a stream of fluid that exhibits the predetermined concentration profile of the reagent.

45. The method of claim 44, wherein the fluid vessel is a capillary tube.

46. The method of claim 44, wherein the predetermined reagent concentration profile exhibits an increasing reagent concentration.

47. The method of claim 44, wherein the predetermined reagent concentration profile exhibits a decreasing reagent concentration.

48. The method of claim 44, further comprising, before step (c), (b′) allowing sufficient time to permit the reagent to diffuse within the cavity to correspond to the predetermined concentration profile of the reagent.

49. The method of claim 44, further comprising, before step (c), (b′) ensuring that the loaded fluid in the vessel forms is free from bubbles.

50. The method for carrying out a chemotactic cellular assay, comprising:
(a) providing a flow passage defined at least in part by a substrate having a cell immobilized on a surface thereof;
(b) forming a concentration gradient of a reagent over the cell by controlled delivery of a fluid containing the reagent in laminar flow through the flow passage and over the target region;
(c) detecting a chemotactic response to the concentration gradient.

51. The method of claim 50, wherein step (c) comprises detecting a change in the position of the cell.

52. The method of claim 50, wherein step (c) comprises detecting a change in the position of a portion of the cell.

53. The method of claim 52, wherein the portion is the nucleus.

54. The method of claim 50, wherein step (c) comprises detecting a change in the shape of the cell.

55. The method of claim 50, wherein step (c) comprises detecting a chemotactic response caused by a change in the position of the cell or a change in the position of a portion of the cell.

56. A device for carrying out a chemotactic cellular assay, comprising:
a flow passage defined at least in part by a substrate having a cell immobilized on a surface thereof;
a means for forming a concentration gradient of a reagent over the cell by controlled delivery of a fluid containing the reagent in laminar flow through the flow passage and over the target region; and
a detector for detecting chemotactic cellular response to the concentration gradient.

57. The device of claim 56, wherein the detector comprises an optical imaging system.

58. The device of claim 57, wherein the detector comprises a microscope.
